(19) United States
(12) Patent Application Publication
(10) Pub. No.: US 2015/0079018 A1
(43) Pub. Date: Mar. 19, 2015

(54) COMPOSITION FOR PREVENTING BACKFLOW OF ODOR AND METHOD FOR PREVENTING BACKFLOW OF ODOR USING THE SAME

(71) Applicant: WATERLESS LABS CO., LTD, Seoul (KR)

(72) Inventor: Steven Joon Han, Seoul (KR)

(21) Appl. No.: 14/394,270

(22) PCT Filed: Apr. 23, 2013
(86) PCT No.: PCT/KR2013/003445

§ 371 Date: Oct. 14, 2014

(30) Foreign Application Priority Data
Apr. 24, 2012 (KR) 10-2012-0042712

(51) Int. Cl. A61L 9/013

(52) U.S. Cl. CPC A61L 9/013
USPC 424/76.7

(57) ABSTRACT

Disclosed is a composition for preventing backflow of odor, produced by mixing a cyclic siloxane organic solvent or a hydrocarbon organic solvent with an aromatic oil that has oil extracted from the nature, wherein the cyclic siloxane organic solvent includes at least one of a cyclic siloxane tetramer, pentamer, or hexamer.

### TECHNICAL FIELD

The present invention relates to a composition for preventing backflow of odor, and more particularly, to a composition for preventing backflow of odor in a urinal, produced by mixing a cyclic siloxane or hydrocarbon organic solvent with an aromatic oil, and a method for preventing backflow of odor using the same.

### BACKGROUND ART

In a conventional toilet, urine is combined with water and bacteria decompose protein contained in the urine in or around a drain, causing unpleasant odor inherent to a bathroom. Moreover, much water is used to flush a small amount of urine, which becomes an environmental problem. To overcome this problem, toilets without water or with a small amount of water are under active development.

However, a waterless urinal or a urinal using a small amount of water causes strong odor due to remaining urine and there are limitations in removing the odor simply by changing the structure of the urinal.

### DISCLOSURE

### Technical Problem

An object of the present invention devised to solve the conventional problem is to provide a composition for preventing backflow of odor. That is, an object of the present invention is to provide a composition produced by mixing a cyclic siloxane or hydrocarbon organic solvent with an aromatic oil, for preventing backflow of odor in a urinal.

It will be appreciated by persons skilled in the art that the objects that could be achieved with the present invention are not limited to what has been particularly described hereinabove and the above and other objects that the present invention could achieve will be more clearly understood from the following detailed description.

### Technical Solution

In an aspect of the present invention, a composition for preventing backflow of odor is produced by mixing a cyclic siloxane organic solvent with an aromatic oil.

In another aspect of the present invention, a composition for preventing backflow of odor is produced by mixing a hydrocarbon organic solvent with an aromatic oil.

The cyclic siloxane organic solvent may include at least one of a cyclic siloxane tetramer, pentamer, or hexamer.

The aromatic oil may have oil extracted from the nature.

The content of the aromatic oil may be 5 to 10 wt %.

The composition may further contain at least one of a disfectant or an antiseptic.

### Advantageous Effects

If a composition for preventing backflow of odor according to the present invention is injected into a urinal or the like, the composition forms a film on remaining urine in the urinal, thereby preventing release of bad odor from the urine in the air and providing a pleasant aroma. As a consequence, the air of a bathroom can be maintained comfortable.

Further, since the composition for preventing backflow of odor according to the present invention is microbiologically degradable, even though the composition flows down a drain pipe along with water used to clean a urinal or along with urine in the urinal, the composition does not cause any environmental problem and thus can be used safely.

It will be appreciated by persons skilled in the art that that the effects that can be achieved through the present invention are not limited to what has been particularly described hereinabove and other advantages of the present invention will be more clearly understood from the following detailed description.

### BEST MODE FOR CARRYING OUT THE INVENTION

The objects and effects of the present invention and technical structures to achieve them will become apparent to those having ordinary skill in the art upon examination of the following embodiments of the present invention described with reference to the attached drawings. A detailed description of known functions or constructions will be omitted lest it should obscure the subject matter of the present invention. Terms used herein are defined in consideration of functions according to the present invention and may be changed according to the intention of a user or an operator or customs.

However, the present invention is not limited to the embodiments described below. Rather, the present invention may be implemented in many other ways. The embodiments of the present invention are provided to make the disclosure of the present invention comprehensive and give a comprehensive scope of the present invention to those skilled in the art. The present invention is defined by the scope of the claims and the definition should be made based on the comprehensive contents of the present specification.

As used in the present disclosure, terms such as “includes” or “may include” refer to the presence of the corresponding component and is not intended to exclude one or more additional components, unless otherwise specified.

Now, a composition for preventing backflow of odor according to an embodiment of the present invention will be described in detail.

According to an embodiment of the present invention, a composition for preventing backflow of odor is produced by mixing a silicone organic solvent with an aromatic oil.

The silicone organic solvent is used for chemicals, cosmetics, and washing. The types of silicone organic solvents include cyclic siloxane tetramer, pentamer, and hexamer. The silicone organic solvent is odorless, non-toxic and has a high boiling point and a low melting point. Further, due to its low viscosity, density less than 1, and low water solubility, when the silicone organic solvent is mixed with water, it forms a layer on the water, thus serving as a separator. Therefore, even though there is a water component as well as an oil component in urinal, the composition may form a composition layer on the components, thus effectively preventing release of odor into the air.

Particularly, a cyclic siloxane pentamer (Decamethylcyclopentasiloxane) is widely used as an organic solvent for washing. The cyclic siloxane pentamer functions to clean by dissolving fat. In addition, the cyclic siloxane pentamer is a basic ingredient for skin care or body care products such as lotion, soap, shampoo, etc. Without suppressing an immune response in the human body, the cyclic siloxane pentamer ensures human safety. What is better, even though the cyclic siloxane pentamer is released into the nature, more than 99% of it is microbiologically degraded. In this sense, the cyclic siloxane pentamer is an eco-friendly material. As the environmental safety of the cyclic siloxane pentamer is clarified in the Material Safety Data Sheets (MSDS), the cyclic siloxane pentamer may be regarded as verified internationally.

The MSDS is a specification that describes the characteristics of chemicals commercially available in the world. When products are shipped, the MSDS is essentially to be affixed.

According to another embodiment of the present invention, a composition for preventing backflow of odor is produced by mixing a hydrocarbon organic solvent with an aromatic oil.

The hydrocarbon organic solvent has been widely used over the past decades. Particularly, a petroleum solvent obtained by refining crude oil is also a hydrocarbon organic solvent. The flash point of the petroleum solvent may be increased by mixing other compounds in the course of manufacturing or by adding distillation. The increased flash point lowers the risk of fire, thus securing safety. In fact, a large number of manufacturers of organic solvents for washing produce and sell organic solvents with high flash points in the same manner. Examples of such an organic solvent product with a high flash point include DF-2000, Rynex, Shell Sol 1400HT, Puredry, Dry slov, etc.

According to another embodiment of the invention, a composition for preventing backflow of odor may be produced by mixing aromatic oils.

The aromatic oils are extracted from plants such as herbs, fruits, beans, flowers, grass, etc. Examples of an aromatic oil include pine oil, lavender oil, chamomile oil, orange oil, lemon oil, lemon grass oil, ylang ylang oil, tea tree oil, bergamot oil, rose oil, etc., which contain fragrant ingredients not harmful to the human body. The aromatic oils are readily available in daily living and easy to purchase in a large amount for commercial purposes.

Conventional fragrant ingredients used for industrial purposes include phthalates or allergic substances, which should be used with caution because they cause asthma and headaches. In contrast, these problems may be overcome by using natural oils extracted from the nature, not harmful to the human body.

When the content of an aromatic oil is about 5-10 wt %, it shows an appropriate property. However, the intensity of fragrance may be readily changed by adjusting the content of the aromatic oil according to customers' preferences.

Further, a composition for preventing backflow of odor according to an embodiment of the present invention may additionally contain a disinfectant and antiseptic. Examples of the disinfectant and antiseptic include Phenoxyethanol, Chlorophenesin, Methylparaben, Ethylparaben, Buthylparaben, Isobuthylparaben, Propylparaben, Imidazolidinylurea, etc., which are used for food or cosmetics groups.

A composition for preventing backflow of odor according to another embodiment of the present invention may be injected in a urinal. For example, it may be injected in a drain trap that connects a urinal to a water pipe. In addition, the composition may be applied to other devices under similar environments. For example, the composition may be applied to a local portion or device that does not allow easy cleaning, such as the inlet of a smelly drain pipe or a waste processor. If the composition is used up due to periodic cleaning or its fragrance is reduced, the composition may be re-injected.

As is apparent from the above description, when an appropriate amount of a composition of the present invention is injected in or coated on a contaminated part, contaminants can be eliminated with oil components contained in a cyclic siloxane organic solvent and an aromatic oil of the composition can provide pleasant fragrance.

Those skilled in the art will appreciate that the present invention may be carried out in other specific ways than those set forth herein without departing from the spirit and essential characteristics of the present invention. For example, those skilled in the art will understand that the material, size, etc. of each component may be changed according to an application field or the disclosed embodiments may be combined or replaced within the scope and spirit of the present invention. The above embodiments are therefore to be construed in all aspects as illustrative and not restrictive. The scope of the invention should be determined by the appended claims and their legal equivalents, not by the above description, and all changes coming within the meaning and equivalency range of the appended claims are intended to be embraced therein.

1. A composition for preventing backflow of odor, produced by mixing a cyclic siloxane organic solvent with an aromatic oil.

2. A composition for preventing backflow of odor, produced by mixing a hydrocarbon organic solvent with an aromatic oil.

3. The composition according to claim 1, wherein the cyclic siloxane organic solvent includes at least one of a cyclic. siloxane tetramer, pentamer, or hexamer.

4. The composition according to claim 1, wherein the aromatic oil has oil extracted from the nature.

5. The composition according to claim 1, wherein the content of the aromatic oil is 5 to 10 wt %.

6. The composition according to claim 1, further including at least one of a disfectant or an antiseptic.

7. The composition according to claim 1, wherein the composition is injected into a urinal to prevent backflow of odor caused by urine.

8. The composition according to claim 2, wherein the aromatic oil has oil extracted from the nature.

9. The composition according to claim 2, wherein the content of the aromatic oil is 5 to 10 wt %.

10. The composition according to claim 2, further including at least one of a disfectant or an antiseptic.

11. The composition according to claim 2, wherein the composition is injected into a urinal to prevent backflow of odor caused by urine.

12. The composition according to claim 3, wherein the composition is injected into a urinal to prevent backflow of odor caused by urine.

13. The composition according to claim 4, wherein the composition is injected into a urinal to prevent backflow of odor caused by urine.

14. The composition according to claim 5, wherein the composition is injected into a urinal to prevent backflow of odor caused by urine.

15. The composition according to claim 6, wherein the composition is injected into a urinal to prevent backflow of odor caused by urine.
